(19) United States
(12) Patent Application Publication
(10) Pub. No.: US 2021/0259887 A1
(43) Pub. Date: Aug. 26, 2021

(54) Pressure-Stabilized Ear Flushing Device Capable of Being Operated with One Hand

(71) Applicants: Haibo Hu, Ningbo (CN); Yonggui Zhang, Ningbo (CN)

(72) Inventors: Haibo Hu, Ningbo (CN); Yonggui Zhang, Ningbo (CN)

(21) Appl. No.: 16/798,369

(22) Filed: Feb. 23, 2020

(51) Int. Cl. A61F 11/00; A61M 3/02

(52) U.S. Cl. CPC A61F 11/002; A61M 3/0279; A61M 2210/0662; A61M 3/0216; A61M 3/0262

(57) ABSTRACT

The present invention relates to a pressure-stabilized ear flushing device capable of being operated with one hand, comprising a spray bottle, a spray gun, a long rod for solution transition, and an adapter and a flushing head provided on the long rod. The flushing bottle is provided with a suction tube. The suction tube is connected with the spray gun. The spray gun and the spray bottle are threaded or snapped. The spray head on the spray gun is connected with a long rod. The end of the long rod is connected with a triangular flushing nozzle or an adapter. The other end of the adapter is connected with a hose flushing head. The device of the present invention improves the existing ear flusher, flushes earwax better, treats ears, and is capable of being operated with one hand, which is convenient for users and patients. The device is equipped with a variety of flushing heads, which is suitable for a wide range of people and is convenient and fast to operate.

### TECHNICAL FIELD

The present invention belongs to the technical field of ear flushing, in particular to a pressure-stabilized ear flushing device capable of being operated with one hand.

### BACKGROUND

Ear flushing is commonly used to remove earwax and treat the ear. A conventional methods for removing earwax generally uses ear spoons, cotton swabs, etc., which is generally effective and easy to hurt the ears. The conventional method further comprises going to the hospital for treatment, which is not convenient. For a conventional ear flushing device, such as squeezing water from a bottle or using a syringe, the water pressure of these devices is related to the operation method and is not stable, and there is no replaceable flushing head, which is likely to cause cross infection. Some flushing devices are connected with the flushing head by a hose to achieve the flushing purpose, but the operation is inconvenient. Some existing ear flushing devices need to pressurize and flush the bottle body or push the syringe piston to achieve the purpose of flushing with a large force, which is not only time-consuming and laborious, but also is changed in the water pressure due to the strength of the hand operation and is unstable. Some flushing devices are connected by hoses. When flushing, both hands are needed for operation, squeezing with one hand, and holding the flushing head with the other hand to align the ear hole. The operation is not convenient. In addition, due to the discharge of flushing sewage, the patient can only operate near the pool, or the patient needs to hold a water basin, which makes the patient feel uncomfortable. Therefore, there is a need for an improved ear flushing device, which can not only make the water pressure stable, but also can be operated with one hand. At the same time, multiple flushing heads can be replaced, the operation is convenient, the use is flexible, and various needs can be met.

### SUMMARY

The object of the present invention is to provide a pressure-stabilized ear flushing device capable of being operated with one hand. The spray gun is provided with reflux prevention and voltage stabilization functions. The entire device is easy to assemble, disassemble and replace, and is capable of being operated with one hand, which is convenient and better to wash earwax or treat ears.

In order to achieve the above object, the technical solution of the present invention is as follows.

A pressure-stabilized ear flushing device capable of being operated with one hand is provided, comprising a spray bottle, a spray gun, a long rod for solution transition, and an adapter and a flushing head provided on the long rod, wherein the flushing bottle is provided with a suction tube, the suction tube is connected with the spray gun, the spray gun and the spray bottle are threaded or snapped, the spray head on the spray gun is connected with a long rod, the end of the long rod is connected with a triangular flushing nozzle or an adapter, and the other end of the adapter is connected with a hose flushing head.

Further, the spray bottle is in a circular shape, a conical shape, or other shapes, not limited in material, and transparent or solid in color; the spray bottle and spray gun are threaded, but not limited to threaded connection; the threaded end of the spray gun is matched with the internal thread of the long rod, and the spray gun and the long rod are naturally sealed after being tightened.

Further, the large head end of the long rod is provided with a first internal thread, and the first internal thread is matched with the external thread of the water outlet end of the spray gun; the small head end is provided with a second internal thread and a first internal rod sealing bevel, and the second internal thread and the first internal rod sealing bevel are sealingly connected with the adapter or the triangular flushing head.

Further, the first end of the adapter is provided with an external thread and a hole sealing bevel, the external thread is connected with the second internal thread of the long rod, and the taper hole is matched and sealed with the sealing bevel of the long rod after the thread is tightened; the second end of the adapter is provided with a third internal thread and a second internal rod sealing bevel; the first end of the hose flushing head is provided with a first external thread and an inner hole sealing bevel; the third internal thread of the adapter is matched with the external thread of the hose flushing head, and the taper hole of the hose flushing head is matched and sealed with the sealing bevel of the adapter after the thread is tightened.

Further, the adapter is split or integral, the material is hard or soft; the adapter is further provided with a water shield in addition to being provided with a thread and a sealing tapered surface connected with the long rod and the hose flushing head; the water shield is independently installed on the adapter or is integrated with the flushing head; the water shield is transparent in color without affecting the line of sight; and the outer diameter of the water shield is larger than the diameter of the flushing head.

Further, the hose flushing head is split, and a hard connector is connected with the hose; or the hose flushing head is integrally formed; the part where the hose flushing head is connected with the adapter is made of hard or soft material, the tube that penetrates into the ear is made of soft and transparent material, such as silicone or PVC; and the length of the hose is set according to the depth of the ear canal, which is 12-18 mm.

Further, one end of the triangular flushing head is provided with a second external thread and a hole sealing bevel, the second external thread is connected with the thread of the long rod, the taper hole is matched and sealed with the sealing bevel of the long rod after the thread is tightened; the triangular flushing head is made of soft material; the triangular flushing head is designed with a large head and a small head, the large head is close to the long rod, and the small head is used to spray ears; and the triangular flushing head is designed with a non-slip thread on the outside.

Further, the water shield outside the adapter is provided with a first opening reflux tank, a second opening reflux tank and a third opening reflux tank which are evenly distributed, and the opening reflux tanks penetrate from the small head end to the large head end; the water shield outside the triangular flushing head is provided with a fourth opening reflux tank, a fifth opening reflux tank, and a sixth opening reflux tank which are evenly distributed, and the opening reflux tanks penetrate from the small head end to the large head end.

Further, the long rod and the adapter are in a fixed structure; the adapter is provided with a large-diameter baffle and an opening reflux tank to prevent the splash of sewage reflux.

Further, the ear wash basin is designed according to the curve in contact with the face and head under the ear; the two side walls of the ear wash basin are provided with a small groove and a large groove.

During the operation of the device of the present invention, the solution to be flushed is put in the spray bottle in advance, the spray gun is mounted on the spray bottle, and the actuator on the spray head is squeezed. The solution is lifted to the inner cavity of the spray gun through the suction tube in the spray bottle and flows to a one-way nozzle. The solution flows into the transition long rod through the nozzle to reach the flushing head, and enters the ear from the flushing head. The flushing sewage can be received by an ear wash basin. During the operation, one hand holds the spray bottle to press and flush, and the other hand holds the ear wash basin to receive the sewage.

The beneficial effect of the present invention is that the device of the present invention improves the existing ear flusher, flushes earwax better, treats ears, and is capable of being operated with one hand, which is convenient for users and patients. The device is equipped with a variety of flushing heads, which is suitable for a wide range of people and is convenient and fast to operate.

### BRIEF DESCRIPTION OF THE DRAWINGS

FIG. 1 is an assembly diagram of an ear flushing device equipped a hose flushing head according to an embodiment of the present invention.

FIG. 2 is an assembly diagram of an ear flushing device equipped with a triangular flushing head according to an embodiment of the present invention.

FIG. 3 is an exploded diagram of an ear flushing device equipped with a hose flushing head according to an embodiment of the present invention.

FIG. 4 is an exploded diagram of an ear flushing device equipped with a triangular flushing head according to an embodiment of the present invention.

FIG. 5 is a cross-sectional diagram of a long rod equipped with a hose flushing head according to an embodiment of the present invention.

FIG. 6 is a cross-sectional diagram of a long rod equipped with triangular flushing head according to an embodiment of the present invention.

FIG. 7 is a schematic diagram of a three-dimensional structure of an adapter according to an embodiment of the present invention.

FIG. 8 is a bottom schematic diagram of a structure of an adapter according to an embodiment of the present invention.

FIG. 9 is a front schematic diagram of a structure of a triangular flushing head according to an embodiment of the present invention.

FIG. 10 is a bottom schematic diagram of a structure of a triangular flushing head according to an embodiment of the present invention.

FIG. 11 is a schematic diagram of a three-dimensional structure of an ear wash basin according to an embodiment of the present invention.

FIG. 12 is a bottom schematic diagram of a structure of an ear wash basin according to an embodiment of the present invention.

### DESCRIPTION OF THE EMBODIMENTS

The specific embodiments of the present invention are described below with reference to the accompanying drawings and embodiments in order to better understand the present invention.

### EMBODIMENT

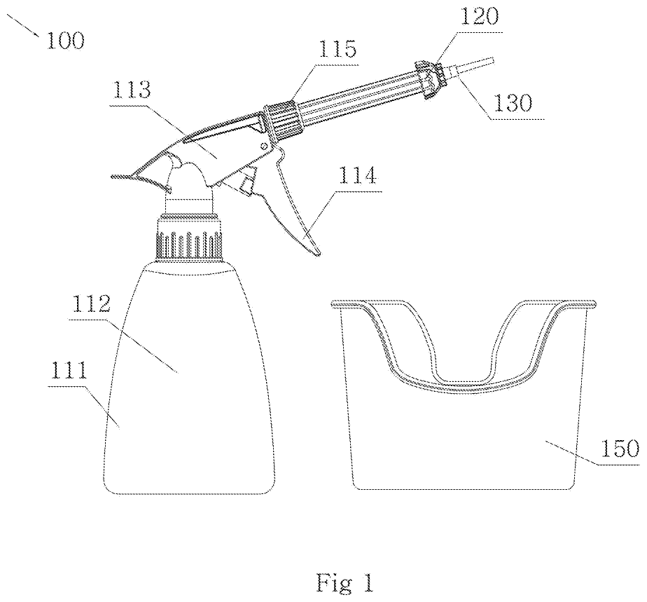

The ear flushing device 100 shown in FIG. 1 comprises a spray bottle 111, a suction tube 112, a spray gun 113, an actuator 114, a long rod 115, an adapter 120, a hose flushing head 130, and an ear wash basin 150. A suction tube 112 is provided in the spray bottle 111. The suction tube 112 is mounted at the bottom of the spray gun 113 in advance, and the spray gun 113 is connected with the spray bottle 111; the water outlet end of the spray gun 113 is connected with the large head end of the long rod 115; the second end of the long rod 115 is connected with one end of the adapter 120, and the second end of the adapter 120 is connected with one end of the hose connector 130.

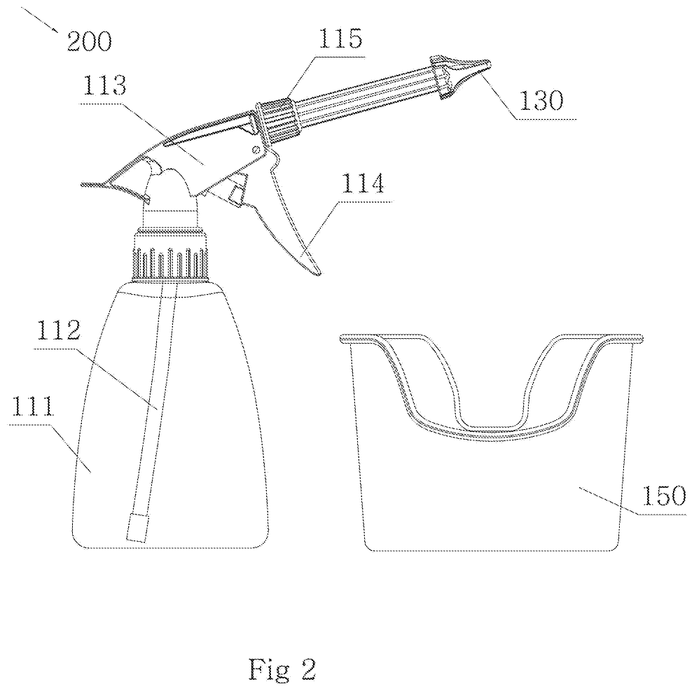

The ear flushing device 200 shown in FIG. 2 comprises a spray bottle 111, a suction tube 112, a spray gun 113, an actuator 114, a long rod 115, a triangular flushing head 140, and an ear wash basin 150. A suction tube 112 is provided in the spray bottle 111. The suction tube 112 is mounted at the bottom of the spray gun 113 in advance, and the spray gun 113 is connected with the spray bottle 111; the water outlet end of the spray gun 113 is connected with the large head end of the long rod 115; and the second end of the long rod 115 is connected with the triangular flushing head 140.

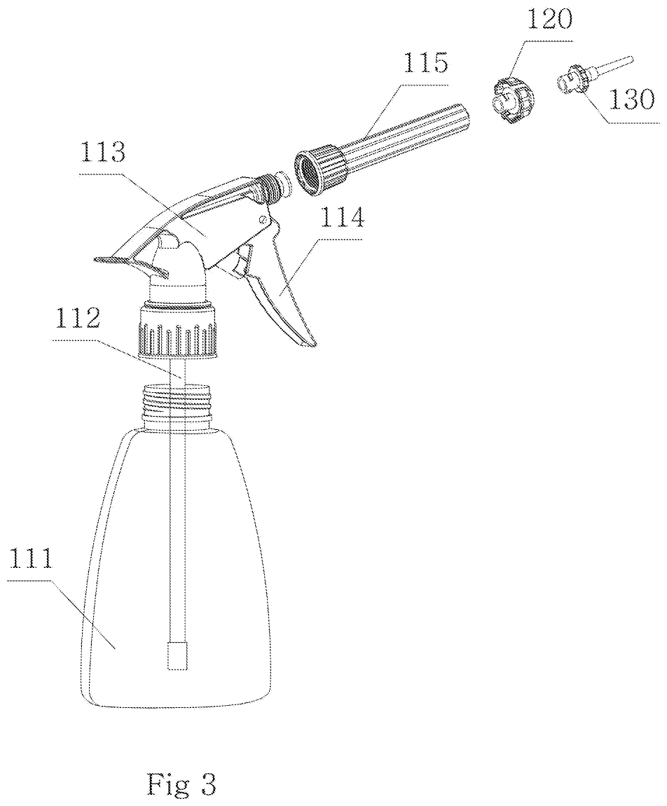

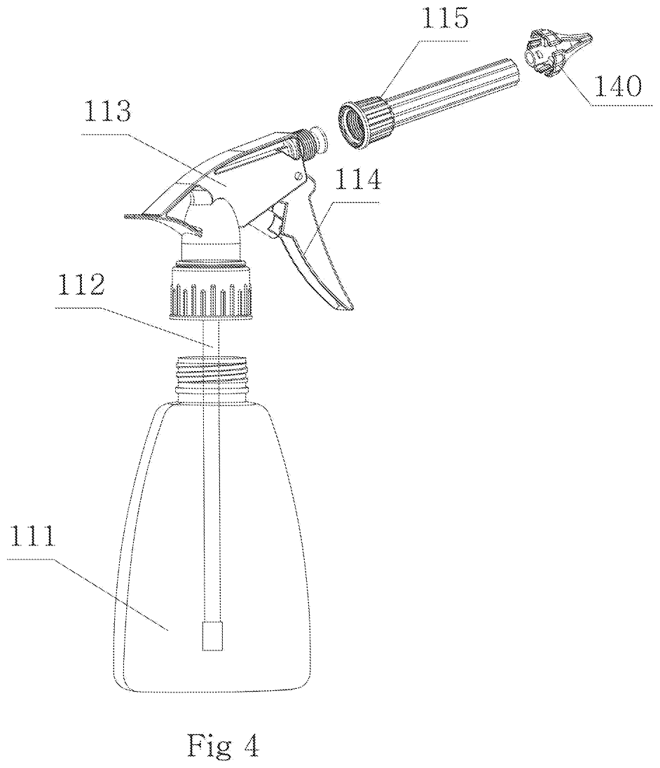

As shown in FIG. 3 and FIG. 4, the spray bottle 111 is in a circular shape, a conical shape, or other shapes, not limited in material, and transparent or solid in color. The amount of liquid can be better observed by the transparent color. The spray bottle 111 and spray gun 113 are threaded, but not limited to threaded connection. There are many types of spray guns 113, and the spraying force can be selected according to needs. Each time the actuator 114 is pressed, the volume and water pressure sprayed by the spray gun 113 each time are the same, which is determined by the internal structure of the spray gun 113. The threaded end of the spray gun 113 is matched with the internal thread of the long rod 115, and the spray gun and the long rod are naturally sealed after being tightened. This is the conventional matching principle of the spray gun structure.

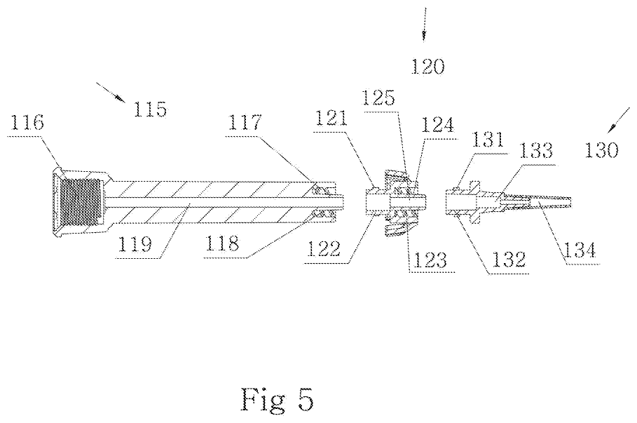

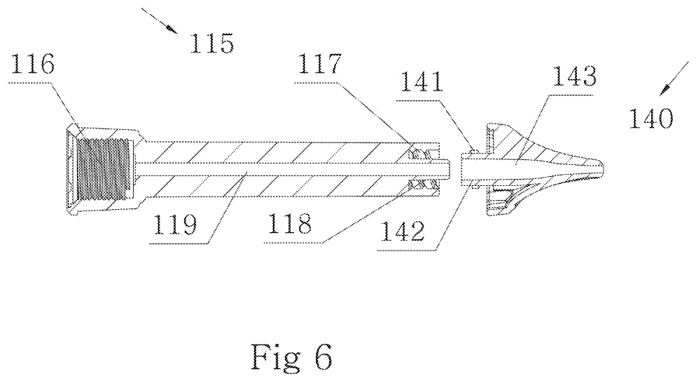

As shown in FIG. 5 and FIG. 6, the large head end of the long rod 115 is provided with a first internal thread 116, and the first internal thread 116 is matched with the external thread of the water outlet end of the spray gun 113. The small head end of the long rod 115 is provided with a second internal thread 117 and a first internal rod sealing bevel 118, and the second internal thread 117 and the first internal rod sealing bevel 118 are sealingly connected with the adapter 120 or the triangular flushing head 140. The long rod 115 is made of hard material, which is not easily deformed. In addition, a non-slip thread is provided on the outside, which is convenient to mount.

As shown in FIG. 5, the first end of the adapter 120 is provided with an external thread 121 and a hole sealing bevel 122, the external thread 121 is connected with the second internal thread 117 of the long rod 115, and the taper hole 122 is matched and sealed with the sealing bevel 118 of the long rod 115 after the thread is tightened. The second end of the adapter 120 is provided with a third internal thread 123 and a second internal rod sealing bevel 124. The first end of the hose flushing head 130 is provided with a first external thread 131 and an inner hole sealing bevel 132. The third internal thread 123 of the adapter 120 is matched with the external thread of the hose flushing head 130, and the taper hole 132 of the hose flushing head 130 is matched and sealed with the sealing bevel 124 of the adapter 120 after the thread is tightened.

The adapter 120 is split or integral, and the material is hard or soft; the adapter 120 is further provided with a water shield in addition to being provided with a thread and a sealing tapered surface connected with the long rod 115 and the hose flushing head 130. The water shield is independently installed on the adapter 120 or is integrated with the flushing head 130. The water shield is transparent in color without affecting the line of sight. The outer diameter of the water shield is larger than the diameter of the flushing head 130. When flushing, the water shield can prevent the splash of reflux sewage, and the sewage can flow out along a diversion trench reserved on the water shield to protect the patient and the environment.

The hose flushing head 130 is split, and a hard connector is connected with the hose; or the hose flushing head is integrally formed. The part where the hose flushing head 130 is connected with the adapter 120 is made of hard or soft material. The tube 134 that penetrates into the ear is made of soft and transparent material, such as silicone or PVC. The tube will not hurt the ear during use, and it is easy to observe the use situation. The tube can be replaced in time if it is dirty or blocked. The length of the hose is set according to the depth of the ear canal, which is generally 12-18 mm, which can be selected according to different people.

As shown in FIG. 6, one end of the triangular flushing head 140 is provided with a second external thread 141 and a hole sealing bevel 142, the second external thread 141 is connected with the thread 117 of the long rod 115, and the taper hole 142 is matched and sealed with the sealing bevel 118 of the long rod 115 after the thread is tightened.

The triangular flushing head 140 is made of soft material, which will not hurt the ear. the triangular flushing head 140 is designed with a large head and a small head, the large head is close to the long rod 115, and the small head is used to spray ears. The large head is designed to prevent the flushing head from entering the ears too much and causing damage to the ear holes. The triangular flushing head 140 is designed with a non-slip thread on the outside, which is easy to operate and connect.

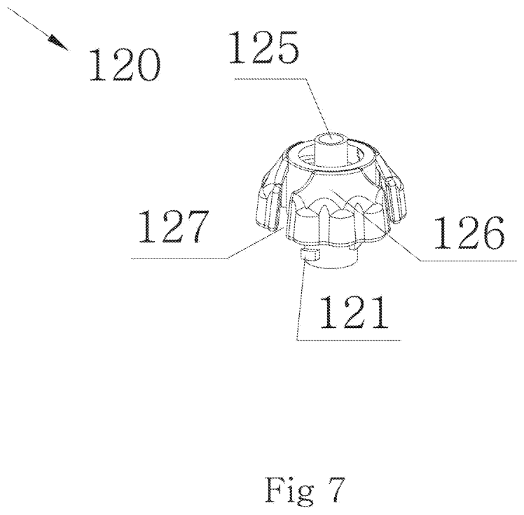

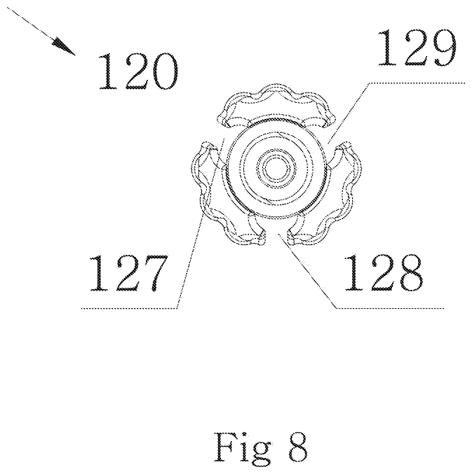

As shown in FIG. 7 and FIG. 8, the water shield outside the adapter 120 is provided with a first opening reflux tank 127, a second opening reflux tank 128 and a third opening reflux tank 129 which are evenly distributed, and the opening reflux tanks penetrate from the small head end to the large head end. When the flushing sewage returns, the sewage can flow out along the opening reflux tank.

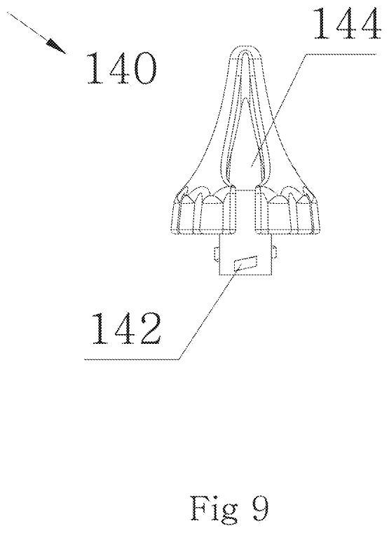

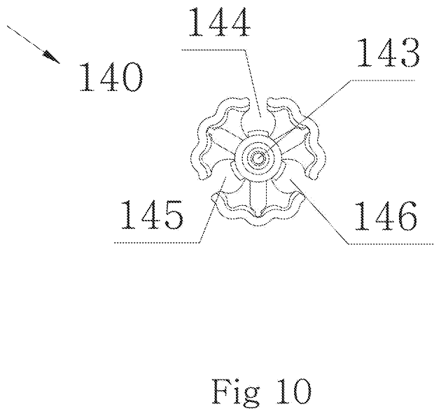

As shown in FIG. 9 and FIG. 10, the water shield outside the triangular flushing head 140 is provided with a fourth opening reflux tank 144, a fifth opening reflux tank 145, and a sixth opening reflux tank 146 which are evenly distributed, and the opening reflux tanks penetrate from the small head end to the large head end. When the flushing sewage returns, the sewage can flow out along the opening reflux tank.

The triangular flushing head 140 and the hose flushing head 130 are integrally replaced as needed. The long rod 115 and the adapter 120 are generally in a fixed structure. The adapter 120 is set to allow flexible replacement of the hose flushing head. In addition, the adapter 120 is provided with a large-diameter baffle 126 to prevent the splash of sewage reflux.

As shown in FIG. 5, when the ear is flushed, liquid flows from the first inner hole 119 of the long rod 115 to the second inner hole 125 of the adapter 120, and then flows to the third inner hole 133 and the fourth inner hole 134 of the hose connector 130, thereby entering the ear canal. As shown in FIG. 6, when the ear is flushed, liquid flows from the first inner hole 119 of the long rod 115 to the inner hole 143 of the triangular flushing head 140, thereby entering the ear canal. The actuator 114 is continuously squeezed so that the water pressure is constant. The liquid is sprayed out from the small hole of the flushing head through the long rod 115, the adapter 120 or the flushing head 130. The aperture at the end is about 1.5 mm, and the water flows in a straight line with a flushing force, which is easier to clean the earwax.

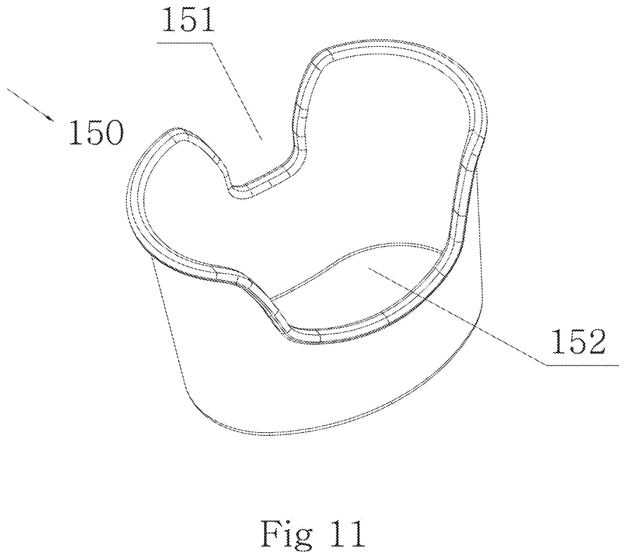

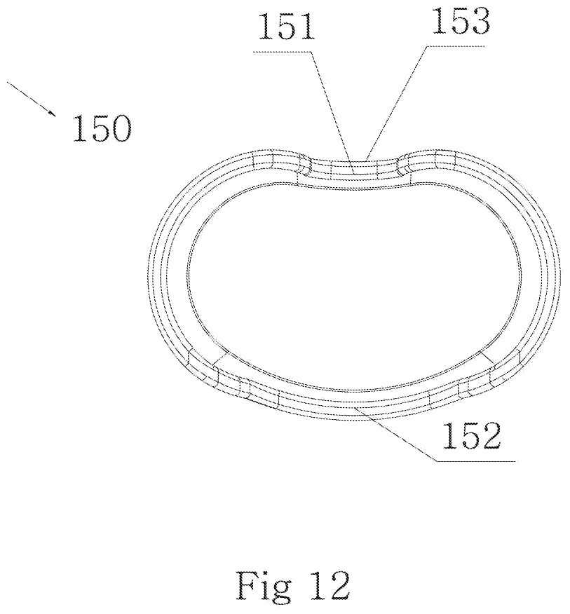

As shown in FIG. 11 and FIG. 12, the ear wash basin 150 is designed according to the curve in contact with the face and head under the ear. The concave curve 153 can fit well on the lower part of the ear. The two side walls of the ear wash basin 150 are provided with a small groove 151 and a large groove 152. When the ear wash basin 150 is used, the small groove 151 is close to the ear and surrounds the ear, leaving ear space, which does not squeeze the ear and is close to the ear at the same time; the large groove 152 is outside, leaving space for the spray gun to move. The ear wash basin 150 is different in size and volume, and can be selected according to actual needs.

Although the present invention uses thread and taper sealed connections in many places, similar to the Luer taper connection mode, the actual product is not limited thereto. Other structural connections can be used, such as tight fitting connection or clamping assistance, so as to achieve the purpose of connecting and sealing. The accessories in the present invention can also be made of various materials, such as the bottle body, the connecting piece, etc., The accessories not only can be made of plastic, but also made of metal. The present invention improves the disadvantages of the conventional ear flushing assembly, and is a complete flushing device that allows patients to remove earwax or treat ears, which is time- and labor-saving, fast and convenient.

The above is the preferred embodiment of the present invention. It should be pointed out that those of ordinary skill in the art can make several improvements and modifications, which are also considered to fall within the protection scope of the present invention, without departing from the principle of the present invention.

What is claimed is:

1. A pressure-stabilized ear flushing device capable of being operated with one hand, comprising a spray bottle, a spray gun, a long rod for solution transition, and an adapter and a flushing head provided on the long rod, wherein the flushing bottle is provided with a suction tube, the suction tube is connected with the spray gun, the spray gun and the spray bottle are threaded or snapped, the spray head on the spray gun is connected with a long rod, the end of the long rod is connected with a triangular flushing nozzle or an adapter, and the other end of the adapter is connected with a hose flushing head.

2. The pressure-stabilized ear flushing device capable of being operated with one hand according to claim 1, wherein the spray bottle is in a circular shape, a conical shape, or other shapes, not limited in material, and transparent or solid in color; the spray bottle and spray gun are threaded, but not limited to threaded connection; the threaded end of the spray gun is matched with the internal thread of the long rod, and the spray gun and the long rod are naturally sealed after being tightened.

3. The pressure-stabilized ear flushing device capable of being operated with one hand according to claim 1, wherein: the large head end of the long rod is provided with a first internal thread, and the first internal thread is matched with the external thread of the water outlet end of the spray gun; the small head end is provided with a second internal thread and a first internal rod sealing bevel, and the second internal thread and the first internal rod sealing bevel are sealingly connected with the adapter or the triangular flushing head.

4. The pressure-stabilized ear flushing device capable of being operated with one hand according to claim 1, wherein the first end of the adapter is provided with an external thread and a hole sealing bevel, the external thread is connected with the second internal thread of the long rod, and the taper hole is matched and sealed with the sealing bevel of the long rod after the thread is tightened; the second end of the adapter is provided with a third internal thread and a second internal rod sealing bevel; the first end of the hose flushing head is provided with a first external thread and an inner hole sealing bevel; the third internal thread of the adapter is matched with the external thread of the hose flushing head, and the taper hole of the hose flushing head is matched and sealed with the sealing bevel of the adapter after the thread is tightened.

5. The pressure-stabilized ear flushing device capable of being operated with one hand according to claim 1, wherein the adapter is split or integral, the material is hard or soft; the adapter is further provided with a water shield in addition to being provided with a thread and a sealing tapered surface connected with the long rod and the hose flushing head; the water shield is independently installed on the adapter or is integrated with the flushing head; the water shield is transparent in color; and the outer diameter of the water shield is larger than the diameter of the flushing head.

6. The pressure-stabilized ear flushing device capable of being operated with one hand according to claim 1, wherein the hose flushing head is split, and a hard connector is connected with the hose; or the hose flushing head is integrally formed; the part where the hose flushing head is connected with the adapter is made of hard or soft material, the tube that penetrates into the ear is made of soft and transparent material, such as silicone or PVC; and the length of the hose is set according to the depth of the ear canal, which is 12-18 mm.

7. The pressure-stabilized ear flushing device capable of being operated with one hand according to claim 1, wherein one end of the triangular flushing head is provided with a second external thread and a hole sealing bevel, the second external thread is connected with the thread of the long rod, the taper hole is matched and sealed with the sealing bevel of the long rod after the thread is tightened; the triangular flushing head is made of soft material; the triangular flushing head is designed with a large head and a small head, the large head is close to the long rod, and the small head is used to spray ears; and the triangular flushing head is designed with a non-slip thread on the outside.

8. The pressure-stabilized ear flushing device capable of being operated with one hand according to claim 1, wherein the water shield outside the adapter is provided with a first opening reflux tank, a second opening reflux tank and a third opening reflux tank which are evenly distributed, and the opening reflux tanks penetrate from the small head end to the large head end; the water shield outside the triangular flushing head is provided with a fourth opening reflux tank, a fifth opening reflux tank, and a sixth opening reflux tank which are evenly distributed, and the opening reflux tanks penetrate from the small head end to the large head end.

9. The pressure-stabilized ear flushing device capable of being operated with one hand according to claim 1, wherein the long rod and the adapter are in a fixed structure; the adapter is provided with a large-diameter baffle and an opening reflux tank.

10. The pressure-stabilized ear flushing device capable of being operated with one hand according to claim 1, wherein the ear wash basin is designed according to the curve in contact with the face and head under the ear; the two side walls of the ear wash basin are provided with a small groove and a large groove.
